(19) United States
(12) Patent Application Publication
(10) Pub. No.: US 2018/0120295 A1
(43) Pub. Date: May 3, 2018

(54) PROCESS FOR SEPARATING NUCLEATED CELLS FROM NON-NUCLEATED RED BLOOD CELLS

(71) Applicant: MESOTEX, INC., Great River, NY (US)

(72) Inventor: Giammaria SITAR, Lavagna (IT)

(73) Assignee: MESOTEX, INC., Great River, NY (US)

(21) Appl. No.: 15/570,764

(22) PCT Filed: Apr. 29, 2016
(86) PCT No.: PCT/US2016/029919

§ 371 Date: Oct. 31, 2017

Related U.S. Application Data
(60) Provisional application No. 62/155,613, filed on May 1, 2015.

(51) Int. Cl. G01N 33/50; B01D 21/01; G01N 33/80

(52) U.S. Cl. CPC G01N 33/5002; G01N 33/80; B01D 21/01

(57) ABSTRACT

The present disclosure provides processes for the separation of nucleated cells from non-nucleated red blood cells, populations of cells obtainable by the processes of the disclosure, and devices and kits useful in the processes of the disclosure.

### 1. CROSS REFERENCE TO RELATED APPLICATIONS

This application claims the priority benefit of U.S. provisional application No. 62/155,613, filed May 1, 2015, the contents of which are incorporated herein in their entireties by reference thereto.

### 2. BACKGROUND

There is significant interest in using rare nucleated cells isolated from blood for non-invasive genetic investigation. Chen et al., 2014, Lab Chip. 14(4):626-645. For example, it is hoped that genetic analysis of fetal nucleated cells isolated from peripheral maternal blood will one day replace invasive procedures, such as amniocentesis and chorionic villus sampling, which are currently used to detect fetal genetic abnormalities. Calabrese et al., 2012, Clin. Genet. 82(2):131-9. There is also interest in using isolated circulating cancer cells in the diagnosis and prognosis of cancer. Harouaka et al., 2014, Pharmacol Ther. 141(2): 209-221.

Separating nucleated cells from the abundant non-nucleated red blood cells (RBCs) found in whole blood is commonly required prior to analysis or use of nucleated cells. U.S. Patent Application Publication No. 2010/0151438 A1. Processes for separating nucleated cells from RBCs by RBC sedimentation have been described in the fields of transfusion and cord blood banking. For example, U.S. Pat. No. 4,111,199 to Djerassi and U.S. Pat. No. 4,765,899 to Wells et al. relate to the field of transfusion hematology and describe processes for removing RBCs from whole blood comprising adding an agent that increases the sedimentation rate of RBCs to whole blood and allowing the RBCs to sediment. Sedimentation has been also described as a way to remove RBCs from umbilical cord blood to reduce sample volume prior to storage. Tsang et al., 2001, Transfusion. 41(3):344-52.

While the sedimentation processes described in the fields of transfusion and cord blood banking are suitable for processing larger volumes of blood that contain large numbers of nucleated cells and large numbers of the subpopulations of cells of interest, they are suboptimal for processing small volume blood samples or for processing blood samples in which nucleated cells to be recovered for downstream use or analysis are rare. First, these processes result in significant nucleated cell loss. For example, Wells et al. reported leukocyte recovery rates of only 73-81. Djerassi reported leukocyte concentrates in which 92-95% of the cells were granulocytes, suggesting that most mononuclear cells, which comprise approximately 40% of all white blood cells in whole blood, were lost. Second, these processes provide only a crude separation of RBCs, i.e., the populations of nucleated cells obtained by the processes contain an undesirable amount of RBCs.

Thus, new processes for separating nucleated cells from non-nucleated red blood cells are needed. Such processes should provide a high yield of nucleated cells and separate substantially all RBCs from the nucleated cells.

### 3. SUMMARY

The present disclosure provides processes useful for separating nucleated cells from non-nucleated red blood cells in a sample. The separated nucleated cells can include rare nucleated cells, such as fetal cells or circulating cancer cells, which can be further purified and/or analyzed by conventional means. In certain embodiments, the processes provide a nucleated cell enriched fraction which contains substantially all of the nucleated cells in the sample. In certain embodiments, the nucleated cell enriched fraction is substantially free of non-nucleated red blood cells. Exemplary processes are described in Section 5.1 and embodiments 1 to 44 below.

The present disclosure also provides enriched cell populations. In certain embodiments, the enriched cell population is a population of nucleated cells which is obtainable by the processes of the disclosure. In some embodiments, the enriched cell population is a nucleated cell enriched fraction that is substantially free of non-nucleated red blood cells. Exemplary enriched cell populations are described in Section 5.2 and embodiments 45 to 46 below.

The present disclosure also provides separation devices for obtaining enriched cell populations and which are useful for carrying out the processes of the disclosure and for providing enriched cell populations of the disclosure. Exemplary separation devices are described in Section 5.3 and embodiments 47 to 50 below.

The present disclosure also provides processes for separating nucleated cells from non-nucleated red blood cells using the separation devices of the disclosure. Exemplary processes are described in Section 5.4 and embodiments 51 to 53.

The present disclosure also provides kits comprising an aggregating agent, optionally together with other reagents and/or a separation device. Exemplary kits are described in Section 5.5 and embodiment 54 below.

### 4. BRIEF DESCRIPTION OF THE DRAWINGS

FIG. 1 is a schematic view of an exemplary separation device of the disclosure.

### 5. DETAILED DESCRIPTION

5.1. Overview

The present disclosure provides processes for separating nucleated cells from non-nucleated red blood cells in a mixture comprising the nucleated cells and non-nucleated red blood cells. The mixture can be formed by combining a sample comprising nucleated cells and non-nucleated red blood cells with an aggregating agent or a solution comprising an aggregating agent. The aggregating agent promotes formation of red blood cell aggregates, known as rouleaux, which have a greater sedimentation rate than nucleated cells. Exemplary aggregating agents are described in Section 5.1.2, below. Without being bound by theory, it is believed that as the rouleaux sediment under the force of local gravity in the lumen of a sedimentation device, they displace a RBC depleted phase upward in the lumen, forming a lower layer enriched in RBCs and an upper phase enriched in nucleated cells. It is further believed, again without being bound by theory, that liquid that is displaced upward as the rouleaux sediment will pull along the slower settling nucleated cells, facilitating separation of the mixture into a nucleated cell enriched fraction and a non-nucleated red blood cell enriched fraction. However, nucleated cells can become entrapped in the rouleaux as they sediment, significantly reducing the yield of nucleated cells in the nucleated cell enriched fraction. Significant loss of nucleated cells can be acceptable in some instances, for example, in the field of transfusion hematology where large volumes of donor blood are available, but is unacceptable when the amount of sample is limited and/or the sample contains a rare cell type of interest, e.g., a fetal cell or a stem cell.

The present disclosure solves this problem by providing a process in which a mixture comprising nucleated cells, non-nucleated red blood cells, and an aggregating agent is separated in the lumen of a container under conditions that allow rouleaux to sediment more quickly than in traditional sedimentation processes. It was discovered that nucleated cell yield is significantly increased when separating the mixture in the lumen of a container sized so that the mixture has a height in the lumen during separation that is reduced as compared to traditional sedimentation methods.

As rouleaux sediment in a mixture comprising nucleated cells, non-nucleated red blood cells, and an aggregating agent, the density of the mixture increases toward the bottom of the lumen because density is hematocrit dependent. As used herein, “hematocrit” refers to the ratio of the volume of non-nucleated red blood cells to a given volume of a mixture containing the non-nucleated red blood cells. The sedimentation velocity of the rouleaux decreases as density increases. It is believed that in traditional sedimentation methods, the upward flow of liquid caused by sedimenting rouleaux becomes insufficient to pull nucleated cells out of the sedimenting rouleaux as the sedimentation velocity of the rouleaux decreases. It is believed, again without being bound by any theory, that when the height of the mixture is reduced as compared to traditional sedimentation methods, the rate of upward liquid flow caused by sedimenting rouleaux exceeds the sedimentation velocity of the nucleated cells for a greater portion of the separation time, allowing for a greater number of nucleated cells to be pulled away from the sedimenting rouleaux. In some embodiments, the average height of the mixture in the lumen is less than 4 cm, less than 3.5 cm, less than 3 cm, less than 2 cm, less than 1.5 cm, or less than 1 cm. In some embodiments, the average height of the mixture in the lumen is 1 cm, 1.5 cm, 2 cm, 2.5 cm, 3 cm, 3.5 cm, or 4 cm. In other embodiments, the average height of the mixture in the lumen is in a range between any pair of the foregoing values, such as 1-4 cm, 1-3 cm, 1.5-2.5 cm, 2-3.5 cm, or 1-2 cm. Preferably, the average height of the mixture in the lumen is 1.5-2 cm.

5.1.1. Mixtures Comprising Nucleated Cells and Non-Nucleated Red Blood Cells

The mixture separated into a nucleated cell enriched fraction and a non-nucleated cell enriched fraction by the processes of the disclosure comprises nucleated cells, non-nucleated red blood cells, and one or more aggregating agents. In some embodiments, the mixture is obtained by combining a sample comprising the nucleated cells and non-nucleated red blood cells with an aggregating agent or a solution comprising the aggregating agent.

The separation process of the disclosure can be performed to separate nucleated cells from non-nucleated cells in blood. The blood can be, for example, peripheral blood (e.g., a peripheral blood sample obtained from a pregnant female, a subject afflicted with a cancer, or a healthy subject) or umbilical cord blood. The blood can be from any mammalian source, e.g., a domesticated animal (such as a cat or dog), livestock (e.g., cattle), a research animal (e.g., a mouse, rat or chimpanzee), and is most preferably human.

The blood can be whole blood (i.e., blood drawn directly from a subject) or processed blood. Processed blood can be whole blood diluted with an aqueous solution or a blood fraction. As used herein, a “blood fraction” is a composition that comprises some, but not all, components of whole blood and can comprise a non-blood component, such as a buffer or cell culture media. In some embodiments, the sample is a blood fraction that has been processed to remove some or all plasma. For example, plasma can be removed from whole blood by centrifuging whole blood to form a pellet containing nucleated cells and non-nucleated red blood cells and removing some or all of the supernatant, which comprises plasma. The pellet can then be resuspended in an aqueous solution to provide a blood fraction that is then separated according to a process of the disclosure. In some embodiments, a blood fraction is prepared by diluting blood with an aqueous solution, centrifuging the diluted blood to form a cell pellet containing nucleated cells and non-nucleated red blood cells, and resuspending the cell pellet in an aqueous solution after removing some of the plasma to provide a blood fraction containing nucleated cells, non-nucleated red blood cells and plasma. In some embodiments, the blood fraction contains at least 5%, at least 10%, at least 20%, at least 30%, at least 40%, at least 50%, or more than 50% of the plasma present in the whole blood used to make the blood fraction. In some embodiments, the blood fraction contains 5-10%, 10-20%, 20-30%, 20-50%, or 50-100% of the plasma present in the whole blood used to make the blood fraction, or any other range bounded by lower and upper limits selected from 5%, 10%, 20%, 30%, 40%, 50%, 60%, 70%, 80%, 90%, and 100%.

Aqueous solutions suitable for use in the processes of the disclosure (e.g., for diluting a sample comprising nucleated cells and non-nucleated red blood cells, preparing a blood fraction, and/or preparing a solution of an aggregating agent) include physiological solutions, i.e., solutions that have a similar pH and osmolarity and/or tonicity as blood, such as tissue culture media. Exemplary physiological solutions include Roswell Park Memorial Institute (RPMI) medium, Dulbecco's Phosphate Buffered Saline, Kreb's-Ringer Biocarbonate Buffer, Puck's Saline, Earle medium, and Hanks balanced salt solution. Plasma and mixtures of plasma and a second physiological solution can also be used as aqueous solutions in the processes of the disclosure.

The processes of this disclosure are particularly suited for separating rare nucleated cells from blood, such as stem cells or circulating cancer cells from adult peripheral and fetal cells from peripheral blood of a pregnant woman, into a nucleated cell enriched fraction that contains most of the rare nucleated cells and a non-nucleated red blood cell enriched fraction that contains most of the non-nucleated red blood cells and few, if any, of the rare nucleated cells. For diagnostic testing, the peripheral blood sample is typically 25-30 mL, particularly from pregnant women to ensure that the fetus is not harmed by the reduced maternal blood volume. The processes of the disclosure also permit improved yield of nucleated cells of interest from samples in which they are more prevalent, such as stem cells umbilical cord blood. The amount of blood obtainable from an umbilical cord is variable, and was found to range from 72 to 275 mL in one recent study. Nunes et al., 2015, Brazilian Journal of Hematology and Hemotherapy 37(1):38-42. The processes of the disclosure can be performed using all or part of a peripheral blood or umbilical cord blood sample, e.g., 10-20 mL, 20-30 mL, 20-50 mL, 50-100 mL, 100-150 mL, or more than 150 mL, if available. The amount of blood used can be selected based on the amount of blood available and the number and/or the type of nucleated cells of interest.

The volume of the mixture separated in the lumen of the container can vary based upon the type of sample used to form the mixture. For example, the volume of a mixture prepared from 25 mL of peripheral blood obtained from a pregnant woman can be one quarter of the volume of a mixture prepared from 100 mL of umbilical cord blood if prepared by the same process. In some embodiments, the volume of the mixture is less than 500 mL, less than 400 mL, less than 300 mL, less than 200 mL, less than 100 mL, less than 75 mL, less than 50 mL, less than 40 mL, less than 30 mL, or less than 25 mL. In some embodiments, the volume of the mixture is 25 mL to 50 mL, 50 mL to 100 mL, 100 mL to 200 mL, or 200 mL to 400 mL.

The amount of time necessary to separate the mixture into a nucleated cell enriched fraction and non-nucleated cell enriched fraction is dependent upon density and height of the mixture, and can be empirically determined by those skilled in the art. The density and height of the mixture are preferably selected so that separation of the mixture into a nucleated cell enriched fraction and a non-nucleated red blood cell enriched fraction is substantially complete in 2 to 15 minutes or even longer. In various embodiments, the separation is complete 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, 5 to 8 minutes, or any other range bounded by lower and upper limits selected from 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes and 15 minutes.

Density of the mixture can be modulated by adjusting hematocrit of the mixture and by the use of aqueous solutions of different densities. Low density mixtures provide faster sedimentation of rouleaux and a greater upward pull of nucleated cells relative to high density mixtures. Hematocrit of the mixture can be modulated, for example, by adjusting hematocrit of the sample comprising the nucleated cells and non-nucleated cells prior to forming the mixture, adjusting the concentration of aggregating agent in the solution of aggregating agent so that more or less of the aggregating agent solution is needed, adding an amount of an aqueous solution to the mixture, or a combination thereof. In some embodiments, the mixture has a hematocrit value that is lower than the hematocrit value of whole blood, e.g., a hematocrit value that is one half of the hematocrit value of whole blood. In some embodiments, the hematocrit of the mixture, measured as the volume percentage of non-nucleated red blood cells in the mixture, is 10-45%, 10-30%, 10-20%, 15-45%, 15-30%, 15-20%, 20-45%, 20-30%, 25%-45%, 20-30%, 5%, 10%, 15%, 20%, 25%, 30%, 35%, 40%, or 45%.

5.1.2. Aggregating Agents

The aggregating agent is an agent that promotes the aggregation of non-nucleated red blood cells, and includes aggregating agents known in the art, such as those described in U.S. Pat. No. 5,482,829 and U.S. Patent Application Publication No. 2004/0142463, each incorporated herein by reference. Exemplary aggregating agents include dextran, hydroxyethyl starch (HES), gelatin, pentastarch, ficoll, gum ararbic, polyvinylpyrrolidone, Ficoll™-Hypaque, Histopaque®, 5-(N-2,3-dihydroxypropylacetamido)-2,4,6-tri-iodo-N, N′-bis(2,3 dihydroxypropyl)isophthalamide (Nycodenz®), Polymorphprep™, nucleic acids, proteins, and other natural or synthetic polymers. In some embodiments, the aggregating agent has a molecular weight of at least 40 kDa, e.g., between about 40 kDa and 2000 kDa, between 40, 50 or 60 kDa as the lower limit and 500 kDa as the upper limit or between 40, 50 or 60 kDa as the lower limit and 150 or 200 kDa as the upper limit, such as 70 kDa. In an embodiment, the aggregating agent is dextran.

The aggregating agent will generally, but not necessarily, be in an aqueous solution when combined with a sample comprising nucleated cells and non-nucleated red blood-cells. Suitable aqueous solutions include those identified in Section 5.1.1. In a preferred embodiment, the aggregating agent is dextran dissolved in RMPI media. In some embodiments, the same aqueous solution is used to prepare the sample comprising the nucleated cells and non-nucleated blood cells and to prepare the solution comprising the aggregating agent. By way of example, a sample comprising nucleated cells and non-nucleated red blood cells can be prepared by diluting an amount of blood with RPMI media, and a solution comprising the aggregating agent dextran can be prepared by dissolving an amount of dextran in RPMI media. The mixture to be separated can then be formed by combining the sample with an amount of the dextran solution.

The concentration of the aggregating agent in the mixture can affect rouleaux formation and sedimentation rates. Suitable concentrations of aggregation agents are described in the art, for example, in U.S. Pat. No. 4,111,199, incorporated herein by reference, and can also be determined empirically. In some embodiments, the amount of aggregating agent in the mixture is 0.1-20%, 0.1-1%, 1-10%, 1-5%, 1%, 2%, 3%, 4%, or 5% (w/v). In a preferred embodiment, the mixture comprises 1% dextran (w/v).

5.2. Enriched Cell Populations

The present disclosure provides populations of nucleated cells and populations of non-nucleated red blood cells. The populations of nucleated cells can comprise rare cell types such as stem cells, circulating cancer cells, or, in maternal blood, fetal nucleated cells (including fetal stem cells).

The population of nucleated cells can comprise a nucleated cell enriched fraction obtained by a process of the disclosure, or comprise some or all of the nucleated cells from such nucleated cell enriched fraction. The nucleated cell enriched fraction is depleted of most non-nucleated red blood cells. In some embodiments, the nucleated cell enriched fraction contains no more than 15%, no more than 10%, no more than 9%, no more than 8%, no more than 7%, no more than 6%, no more than 5%, no more than 4%, no more than 3%, no more than 2%, or no more than 1% of the non-nucleated red blood cells in the mixture used to make the nucleated cell enriched fraction. The nucleated cell enriched fraction comprises most of the nucleated cells in the mixture. In some embodiments, the nucleated cell enriched fraction contains at least 80%, at least 85%, at least 90%, at least 95%, at least 96%, at least 97%, at least 98%, at least 99%, or greater than 99% of the nucleated cells in the mixture. In some embodiments, the viability of the nucleated cells in the nucleated cell enriched fraction is greater than 90%, greater than 95%, greater than 96%, greater than 97%, greater than 98%, or greater than 99%.

A population of nucleated cells obtained by a process described herein can be further depleted of non-nucleated red blood cells by subjecting the population to one, two, three, four, or more separations according to a process described herein. When repeating the separation step, the nucleated cell enriched fraction obtained from the first separation can be used to form the mixture for the second separation.

The population of non-nucleated red blood cells can comprise a non-nucleated red blood cell enriched fraction obtained by a process of the disclosure, or comprise some or all of the non-nucleated red blood cells from such non-nucleated red blood cell enriched fraction. In some embodiments, the non-nucleated red blood cell enriched fraction contains at least 80%, at least 85%, at least 90%, at least 95%, at least 96%, at least 97%, at least 98%, at least 99%, or 100% of the non-nucleated red blood cells in the mixture used to make the non-nucleated red blood cell enriched fraction. In some embodiments, the non-nucleated red blood cell enriched fraction contains no more than 20%, no more than 15%, no more than 10%, no more than 5%, no more than 4%, no more than 3%, no more than 2%, or no more than 1% of the nucleated cells in the mixture.

5.3. Devices for Separating Nucleated Cells from Non-Nucleated Red Blood Cells

The present disclosure provides separation devices for separating nucleated cells from non-nucleated red blood cells. The separation devices described herein are containers that can be used in the processes described herein and can be used to provide an enriched cell population, e.g., a nucleated cell enriched fraction.

The separation devices comprise a lumen for separating a mixture comprising nucleated cells and non-nucleated red blood cells. In a preferred embodiment, the lumen comprises a cylindrical section, although non-cylindrical sections of other geometries are also envisioned, e.g., a polyhedral section formed by connected polygons. In some embodiments, the cylindrical or non-cylindrical section is connected at its bottom end to a funnel-shaped section, e.g., a conical-shaped section, and/or connected at its top end to an inverted funnel-shaped section. The separation device can have one or more inlet/outlet ports that allow for the introduction and/or removal of liquid from the lumen, preferably located at the top and bottom of the lumen. When inlet/outlet ports are present, flow deflectors can be positioned within the lumen to deflect fluid introduced through an inlet/outlet port to prevent mixing of fluids within the lumen.

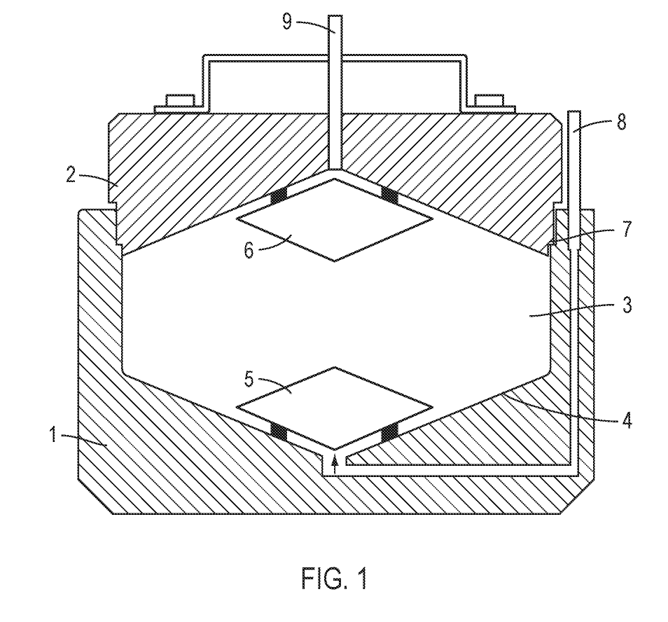

An exemplary separation device is shown in FIG. 1. The separation device shown in FIG. 1 comprises two cylindrical parts (1,2) made, e.g., of transparent polycarbonate. Provided therein is a cylindrical chamber (3) whose bottom is conically shaped internally (4). Above the cylindrical chamber a conical flow-deflecting device (5) is situated. A cylindrical cover (2) whose internal surface is also conically shaped and provided with a conical flow-deflecting device (6) is also provided in this embodiment. The cover and the bottom part of the chamber can be attached to each other via screws and can be sealed via an O-ring (7). The flow deflecting device(s) is/are preferably arranged and fitted in such a way that a liquid flowing in or out (at the top and bottom) must flow through the narrow gap between the flow-deflecting device and the conical chamber wall. The initially high flow velocity is thus reduced, thus allowing the chamber to, e.g., be filled without disturbance. The inlet/outlet ports are preferably tube connections at the center of the chamber (8, 9).

An appropriate separation device for use in a process of the disclosure can be sized based upon the volume of the mixture to be separated and the desired height of the mixture in the lumen. In some embodiments, the separation device is sized so that the volume of a mixture to be separated in the lumen has a height of 4 cm, less than 4 cm, less than 3.5 cm, less than 3 cm, less than 2 cm, less than 1.5 cm, or less than 1 cm. In some embodiments, the separation device is sized so that the average height of the mixture in the lumen is 1 cm, 1.5 cm, 2 cm, 2.5 cm, 3 cm, 3.5 cm, or 4 cm. In other embodiments, the separation device is sized so that the average height of the mixture in the lumen is 1-4 cm, 1-3 cm, or 1-2 cm. Preferably, the separation device is sized so that the average height of the mixture in the lumen is 1.5-2 cm.

For separation of a mixture in a cylindrical section of a lumen, the diameter of an

$$d = 2 \sqrt{\frac{V}{π h}} ,$$

appropriately sized lumen can calculated by the following formula: where V is the volume of the mixture and h is the desired height of the mixture. By way of example, if using a separation device as shown in FIG. 1 to separate a 50 mL mixture with a desired height of no more than 2 cm, the diameter of the lumen should be at least about 5.6 cm.

For mixtures between 25 and 80 mL, cylinder diameters between 5 and 10 can be suitable. For mixtures between 20 and 60 mL, in particular between 45 and 55 mL, diameters of more than 5 cm, such as between 6 cm and 12 cm, between 7 cm and 9 cm, or 8 cm, can be particularly suitable. For larger volume mixtures, e.g., between 80 mL and 250 mL, cylinder diameters between 10 and 20 cm can be suitable. In some embodiments, separation devices of the disclosure comprise a lumen having a cylindrical section with a diameter of 1 to 20 cm, 3 to 8 cm, 4 to 9 cm, 5 to 20 cm, 5 to 10 cm, 6 to 12 cm, 7 to 14 cm, 8 to 12 cm, 8 to 16 cm, 10 to 15 cm, 10 to 20 cm, and in specific embodiment, the diameter is 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm.

5.4. Separation Processes

When using a separation device as described herein in a process of the disclosure, a heavy liquid can be added to the bottom of the lumen of the separation device prior to separation of the mixture comprising nucleated cells, non-nucleated red blood cells, and an aggregating agent. A “heavy liquid” when used in a process for separating a nucleated cell enriched fraction and a non-nucleated red blood cell enriched fraction from a mixture comprising nucleated cells, non-nucleated red blood cells, and an aggregating agent means a liquid having a density that is greater than the density of the liquid in the mixture comprising nucleated cells, non-nucleated red blood cells, and an aggregating agent. In some embodiments, the heavy liquid is a water immiscible liquid. The heavy liquid can have a density of at least 1.05 g/mL, at least 1.1 g/mL, least 1.2 g/mL, at least 1.5 g/mL, 1.75 g/mL or at least 2 g/mL, and can range up to 2.5 g/mL, 3 g/mL or even greater. In particular embodiments, the density ranges between any pair of the foregoing values, e.g., 1.05 g/mL to 1.1 g/mL, 1.05 g/mL to 1.2 g/mL, 1.05 g/mL to 1.5 g/mL, 1.5 g/mL to 2.5 g/mL, 1.2 g/mL to 2 g/mL, and so on and so forth. Suitable heavy liquids include heptacosafluorotributylamine (e.g., Fluorinert™ FC-43, 3M) and Ficoll solutions (e.g., Ficoll solutions having a density of 1.077 g/mL or 1.085 g/mL). The heavy liquid can be introduced to the lumen through an inlet/outlet port, if present. If the lumen has a lower funnel-shaped section, the amount of heavy liquid preferably fills at least the lower funnel-shaped section, and more preferably fills the entire lumen if an inlet/outlet port is present in the lower funnel-shaped section. If the heavy liquid does not fill then entire lower funnel-shaped section, the mixture, when introduced to the lumen, will have different heights at the periphery than in the center and the average height can need to be calculated. Subsequent to introducing the heavy liquid, the mixture is introduced to the lumen on top of the heavy liquid. If the lumen was filled completely with heavy liquid, heavy liquid is allowed to drain from the inlet/outlet port in the lower funnel shaped section as the mixture is introduced to the lumen. The mixture is then allowed to separate into a nucleated cell enriched fraction and a non-nucleated red blood cell enriched fraction in batch, under local gravity.

Following separation, the nucleated cell enriched fraction and/or the non-nucleated red blood cell enriched fraction can be collected from the separation device. If the separation device has an inlet/outlet port at the top of the lumen and an inlet/outlet port at the bottom of the lumen, the nucleated cell enriched fraction can be collected from the top inlet/outlet port by introducing additional heavy liquid through the bottom inlet/outlet port, thereby allowing the nucleated cell enriched fraction to be collected without significantly disturbing the interface between the nucleated cell enriched fraction and the non-nucleated red blood cell enriched fraction. Flow deflectors, when present, help to prevent mixing at the interface between the nucleated cell enriched fraction and the non-nucleated cell enriched fraction.

5.5. Kits

The present disclosure further provides kits comprising an aggregating agent or a solution comprising an aggregating agent, such as the aggregating agents described in Section 5.1.2, above. The kits can also include one or more aqueous solutions suitable for carrying out a process of the disclosure, such as those described in Section 5.1.1, above. The kit can also include a separation device as described in Section 5.3, above. In some embodiments, the kit comprises an aggregating agent or a solution comprising an aggregating agent and an aqueous solution. In some embodiments, the kit comprises an aggregating agent or a solution comprising an aggregating agent and a separation device. In some embodiments, the kit comprises an aggregating agent or a solution comprising an aggregating agent, an aqueous solution, and a separation device.

### 6. EXEMPLARY SEPARATION PROTOCOL

The following separation protocol can be used to obtain a nucleated cell enriched fraction from whole blood.
1. Dilute an amount of blood with an aqueous solution.
2. Centrifuge the diluted blood to form a cell pellet containing nucleated cells and non-nucleated red blood cells and remove some or all of the platelet rich plasma.
3. Resuspend the cell pellet in an aqueous solution.
4. Add a pre-made solution of dextran to the resuspended cells to form a mixture containing, e.g., dextran at a final concentration of 1% (w/v).
5. Add a volume of the mixture to a sedimentation separation device prefilled with heavy liquid to attain a mixture column height of 1.5-2 cm, while simultaneously draining a volume of heavy liquid from the device equal to the volume of the mixture.
6. Allow the mixture to separate into a nucleated cell enriched fraction and a non-nucleated cell enriched fraction.
7. Collect the nucleated cell enriched fraction from the sedimentation separation device.

### 7. EXAMPLES

### 7.1. Example 1

### Separation of Nucleated Cells from Maternal Blood

A separation device of the type shown in FIG. 1 having a lumen 8 cm in diameter was filled with Fluorinert™ FC-43. 25 mL of peripheral blood obtained from a pregnant female was combined with 25 mL of RPMI media comprising 2% dextran (w/v) to provide a mixture having a final dextran concentration of 1%. The mixture was introduced to the lumen of the separation device through the top inlet/outlet port on top of the FC-43 down to the cylindrical part of the separation device and allowed to separate under local gravity into a nucleated cell enriched fraction and a non-nucleated red blood cell enriched fraction for 9 minutes. The nucleated cell enriched fraction was then collected from the top inlet/outlet port by introducing additional FC-43 into the lumen through the bottom inlet/outlet port.

A second separation was performed using the nucleated cell enriched fraction to deplete the sample of remaining non-nucleated red blood cells. After the first separation, the nucleated cell enriched fraction was mixed with RPMI medium containing 1% dextran and introduced again into the lumen of the separation device (after removal of the red blood cells of the first separation) and allowed to sediment under local gravity, to obtain a nucleated cell enriched fraction almost entirely free of red blood cells.

The percent recovery of nucleated cells and non-nucleated red blood cells in the nucleated cell enriched fraction following the first and second separations is shown below in Table 1.

TABLE 1
Cell recovery from maternal blood subjected
to two cycles of separation

| | Percent recovery in the | Percent recovery in the | |
|---|---|---|---|
| | NC enriched fraction | NC enriched fraction | |
| | after one separation | after two separations | p value |
| RBCs | 4.7 ± 1.8 | 1.6 ± 1.2 | <0.005 |
| NCs | 93.6 ± 6.4 | 98.6 ± 5.8 | <0.005 |
| NC viability | 98.7 ± 1.6 | 99.1 ± 1.3 | <0.005 |

### 8. SPECIFIC EMBODIMENTS, CITATION OF REFERENCES

Various aspects of the present disclosure are described in the embodiments set forth in the following numbered paragraphs.

1. A process for separating nucleated cells from non-nucleated red blood cells, comprising:
a) separating a mixture comprising nucleated cells, non-nucleated red blood cells, and an aggregating agent into a nucleated cell enriched fraction and a non-nucleated red blood cell enriched fraction in a lumen of a container at local gravity, wherein the separating is performed in batch, and wherein
i. the average height of the mixture in the lumen is no more than 4 cm; and/or
ii. the average height of the mixture in the lumen is selected to provide a non-nucleated red blood cell enriched fraction that contains at least 80% of the non-nucleated red blood cells in the mixture and/or no more than 20% of the nucleated cells in the mixture after no more than 3 rounds, no more than 2 rounds or no more than one round of separation; and
b) optionally repeating step (a) one or more times,

optionally wherein step (a) comprises maintaining the mixture at local gravity until the mixture separates into a nucleated cell enriched fraction and a non-nucleated red blood cell enriched fraction, optionally for 2 to 15 minutes,

thereby separating nucleated cells from non-nucleated red blood cells.

2. The process of embodiment 1, wherein the average height of the mixture in the lumen is no more than 4 cm, no more than 3.5 cm, no more than 3 cm, no more than 2.5 cm, no more than 2 cm, or no more than 1.5 cm.

3. The process of embodiment 1 or embodiment 2, wherein the average height of the mixture in the lumen is no more than 1 cm.

4. The process of any one of embodiments 1 to 3, wherein the average height of the mixture in the lumen is at least 0.5 cm or at least 1 cm.

5. The process of any one of embodiments 1 to 4, wherein the volume of the mixture is less than 500 mL, less than 400 mL, less than 300 mL, less than 200 mL, less than 100 mL, less than 75 mL, less than 50 mL, less than 40 mL, less than 30 mL, or less than 25 mL.

6. The process of any one of embodiments 1 to 5, wherein the volume of the mixture is at least 5 mL, at least 10 mL, at least 20 mL or at least 25 mL.

7. The process of any one of embodiments 1 to 4, wherein the volume of the mixture is 25 mL to 50 mL, 50 mL to 100 mL, 100 mL to 200 mL, or 200 mL to 400 mL.

8. The process of any one of embodiments 1 to 7, wherein the non-nucleated red blood cell enriched fraction contains at least 80%, at least 85%, at least 90%, t least 95%, at least 96%, at least 97%, at least 98%, at least 99%, or 100% of the non-nucleated red blood cells in the mixture.

9. The process of any one of embodiments 1 to 8, wherein the non-nucleated red blood cell enriched fraction contains no more than 20%, no more than 15%, no more than 10%, no more than 5%, no more than 4%, no more than 3%, no more than 2%, or no more than 1% of the nucleated cells in the mixture.

10. The process of any one of embodiments 1 to 9, wherein the aggregating agent is dextran, hydroxyethyl starch (HES), gelatin, pentastarch, ficoll, gum ararbic, polyvinylpyrrolidone, 5-(N-2,3-dihydroxypropylacetamido)-2,4,6-tri-iodo-N,N′-bis(2,3 dihydroxypropyl)isophthalamide or any combination thereof.

11. The process of any one of embodiments 1 to 10, wherein the mixture is the product of a process comprising combining the aggregating agent or a solution comprising the aggregating agent and a sample comprising the nucleated cells and the non-nucleated red blood cells.

12. The process of embodiment 11, further comprising a step of forming the mixture.

13. The process of embodiment 11 or embodiment 12, wherein the sample is a previously prepared nucleated cell enriched fraction.

14. The process of embodiment 11 or embodiment 12, wherein the sample comprises blood.

15. The process of embodiment 11 or embodiment 12, wherein the sample comprises a blood fraction.

16. The process of embodiment 15, wherein the blood fraction contains at least 5%, at least 10%, at least 20%, at least 30%, at least 40%, at least 50%, or more than 50% of the plasma present in an amount of whole blood used to make the blood fraction.

17. The process of embodiment 15, wherein the blood fraction contains 5-10%, 10-20%, 20-30%, 20-50%, or 50-100% of the plasma present in an amount of whole blood used to make the blood fraction.

18. The process of any one of embodiments 15 to 17, wherein the blood fraction is the product of a process comprising:
a) optionally, diluting blood with an aqueous solution;
b) centrifuging blood or the diluted blood from step (a) to obtain a cell pellet; and
c) optionally, resuspending the pellet in an aqueous solution, which aqueous solution has the same composition as the aqueous solution of step (a) or has a different composition from the aqueous solution of step (a),

thereby forming the blood fraction.

19. The process of embodiment 18, comprising a step of forming the blood fraction.

20. The process of embodiment 11 or embodiment 12, wherein the sample comprises blood diluted with an aqueous solution.

21. The process of any one of embodiments 18 to 20, wherein the aqueous solution comprises plasma, a cell culture medium, a buffered solution, or a combination thereof.

22. The process of embodiment 21, wherein the cell culture medium is Roswell Park Memorial Institute (RPMI) medium, Earle medium, or Hanks balanced salt solution.

23. The process of any one of embodiments 1 to 22, wherein the mixture is isotonic to red blood cells.

24. The process of any one of embodiments 14 to 23, wherein the blood is peripheral blood.

25. The process of embodiment 24, wherein the blood is from a pregnant female, umbilical cord blood, blood obtained from a subject afflicted with a cancer, or blood obtained from a healthy individual.

26. The process of any one of embodiments 1 to 25, wherein the nucleated cells comprise rare nucleated cells.

27. The process of embodiment 26, wherein the rare nucleated cells comprise stem cells or cancer cells.

28. The process of embodiment 26, wherein blood is peripheral blood from a pregnant female and the rare nucleated cells comprise fetal cells.

29. The process of any one of embodiments 1 to 28, further comprising a step of removing all or part of the nucleated cell enriched fraction from the lumen of the container.

30. The process of embodiment 29, comprising removing all or part of the nucleated cell enriched fraction from the lumen of the container after the mixture separates for 2 to 15 minutes, 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, 5 to 8 minutes.

31. The process of any one of embodiments 1 to 30, further comprising a step of removing all or part of the non-nucleated red blood cell enriched fraction from the lumen of the container.

32. The process of embodiment 31, comprising removing all or part of the non-nucleated red blood cell enriched fraction from the lumen of the container after the mixture separates for 2 to 15 minutes, 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, 5 to 8 minutes.

33. The process of any one of embodiments 1 to 32, wherein the lumen of the container has a fixed volume.

34. The process of any one of embodiments 1 to 33, wherein the lumen of the container comprises a cylindrical section or a polyhedral section.

35. The process of any one of embodiments 1 to 34, wherein the lumen of the container comprises a funnel shaped section.

36. The process of any one of embodiments 1 to 34, wherein the lumen of the container comprises a cylindrical section or polyhedral section joined (a) at the bottom end to a funnel shaped section, (b) at one the top end to an inverted funnel shaped section, or (c) at the bottom end to a funnel shaped section and at the top end to an inverted funnel shaped section.

37. The process of any one of embodiments 1 to 36, wherein container comprises one or more inlet/outlet ports operably connected to the lumen of the container.

38. The process of embodiment 37, wherein the container further comprises one or more flow deflectors positioned within the lumen of the container to allow for the deflection of a fluid introduced into the lumen of the container through the one or more of the inlet/outlet ports.

39. The process of embodiment 37 or embodiment 38, further comprising introducing a heavy liquid into the lumen of the container through a first inlet/outlet port positioned at the bottom of the lumen until all or part of the nucleated cell enriched fraction is forced out of the lumen through a second inlet/outlet port positioned at the top of the lumen.

40. The process of embodiment 39, wherein an amount of the heavy liquid is present in the lumen of the container during the separation of the mixture.

41. The process of embodiment 39 or embodiment 40, wherein the heavy liquid comprises heptacosafluorotributylamine, Ficoll 1.077 g/mL, or Ficoll 1.085 g/mL.

42. The process of embodiment 40 or embodiment 41, wherein the mixture is introduced into the lumen of the container after the amount of the heavy liquid is introduced into the container.

43. The process of embodiment 42, comprising a step of introducing the amount of the heavy liquid into the container before introducing the mixture into the lumen of the container.

44. The process of any one of embodiments 1 to 43, comprising a step of introducing the mixture into the lumen of the container.

45. A nucleated cell enriched fraction obtained by the process of any one of embodiments 1 to 44.

46. A non-nucleated red blood cell enriched fraction obtained by the process of any one of embodiments 1 to 44.

47. A separation device suitable for obtaining the nucleated cell enriched fraction of embodiment 45.

48. A separation device suitable for obtaining the non-nucleated red blood cell enriched fraction of embodiment 46.

49. The separation device of embodiment 47 or embodiment 48, comprising a container having a lumen and one or more inlet/outlet ports operably connected to the lumen of the container, optionally wherein:
a) the separation device optionally has the features of a device according to FIG. 1; and/or
b) the lumen comprises a cylindrical section with a diameter of 1 to 20 cm, 3 to 8 cm, 4 to 9 cm, 5 to 20 cm, 5 to 10 cm, 6 to 12 cm, 7 to 14 cm, 8 to 12 cm, 8 to 16 cm, 10 to 15 cm, 10 to 20 cm, or a diameter of 5.6 cm.

50. The separation device of embodiment 49, in which the lumen comprises a cylindrical section and a funnel shaped section.

51. A process for separating nucleated cells from non-nucleated red blood cells, comprising:
a) introducing a mixture comprising nucleated cells, non-nucleated red blood cells, and an aggregating agent into the lumen of a container of a separation device according to embodiment 49 or 50;
b) maintaining the mixture at local gravity until the mixture separates into a nucleated cell enriched fraction and a non-nucleated red blood cell enriched fraction;
c) optionally, recovering one or both fractions; and
d) optionally repeating step (a), step (b) and optionally step (c) one or more times.

52. The process of embodiment 51, wherein the mixture introduced into the lumen in a volume that reaches a height of 1-4 cm, 1-3 cm, or 1-2 cm in the lumen.

53. The process of embodiment 52, wherein the mixture introduced into the lumen in a volume that reaches a height of 1.5-2 cm in the lumen.

54. A kit for use in a process for separating nucleated cells from non-nucleated red blood cells, comprising:
a) an aggregating agent and/or a solution comprising an aggregating agent;
b) an aqueous solution;
c) a separation device; or
d) any combination thereof.

All cited references are incorporated herein by reference in their entirety and for all purposes to the same extent as if each individual publication, patent or patent application was specifically and individually indicated to be incorporated herein by reference in its entirety for all purposes.

Many modifications and variations of this disclosure can be made without departing from its spirit and scope, as will be apparent to those skilled in the art. The specific embodiments described are offered by way of example only, and the disclosure is to be limited only by the terms of the claims along with the full scope of equivalents to which such claims are entitled.

What is claimed is:

1. A process for separating nucleated cells from non-nucleated red blood cells, comprising:
a) separating a mixture comprising nucleated cells, non-nucleated red blood cells, and an aggregating agent into a nucleated cell enriched fraction and a non-nucleated red blood cell enriched fraction in a lumen of a container at local gravity, wherein the separating is performed in batch, and wherein
i. the average height of the mixture in the lumen is no more than 4 cm; and/or
ii. the average height of the mixture in the lumen is selected to provide a non-nucleated red blood cell enriched fraction that contains at least 80% of the non-nucleated red blood cells in the mixture and/or no more than 20% of the nucleated cells in the mixture after no more than 3 rounds, no more than 2 rounds or no more than one round of separation; and
b) optionally repeating step (a) one or more times,
optionally wherein step (a) comprises maintaining the mixture at local gravity until the mixture separates into a nucleated cell enriched fraction and a non-nucleated red blood cell enriched fraction, optionally for 2 to 15 minutes,
thereby separating nucleated cells from non-nucleated red blood cells.

2. The process of claim 1, wherein the average height of the mixture in the lumen is (a) no more than 4 cm, no more than 3.5 cm, no more than 3 cm, no more than 2.5 cm, no more than 2 cm, no more than 1.5 cm and/or (b) no more than 1 cm and/or at least 0.5 cm or at least 1 cm.

3. The process of claim 1, wherein the volume of the mixture is (a) less than 500 mL, less than 400 mL, less than 300 mL, less than 200 mL, less than 100 mL, less than 75 mL, less than 50 mL, less than 40 mL, less than 30 mL, or less than 25 mL and/or (b) at least 5 mL, at least 10 mL, at least 20 mL or at least 25 mL.

4. The process of claim 1, wherein the volume of the mixture is 25 mL to 50 mL, 50 mL to 100 mL, 100 mL to 200 mL, or 200 mL to 400 mL.

5. The process of claim 1, wherein the non-nucleated red blood cell enriched fraction contains at least 80%, at least 85%, at least 90%, at least 95%, at least 96%, at least 97%, at least 98%, at least 99%, or 100% of the non-nucleated red blood cells in the mixture.

6. The process of claim 1, wherein the non-nucleated red blood cell enriched fraction contains no more than 20%, no more than 15%, no more than 10%, no more than 5%, no more than 4%, no more than 3%, no more than 2%, or no more than 1% of the nucleated cells in the mixture.

7. The process of claim 1, wherein the aggregating agent is dextran, hydroxyethyl starch (HES), gelatin, pentastarch, ficoll, gum ararbic, polyvinylpyrrolidone, 5-(N-2,3-dihydroxypropylacetamido)-2,4,6-tri-iodo-N,N′-bis(2,3 dihydroxypropyl)isophthalamide or any combination thereof.

8. The process of claim 1, wherein the mixture is the product of a process comprising combining the aggregating agent or a solution comprising the aggregating agent and a sample comprising the nucleated cells and the non-nucleated red blood cells.

9. The process of claim 8, further comprising a step of forming the mixture.

10. The process of claim 8, wherein the sample is a previously prepared nucleated cell enriched fraction.

11. The process of claim 8, wherein the sample comprises blood.

12. The process of claim 8, wherein the sample comprises a blood fraction.

13. The process of claim 12, wherein the blood fraction contains at least 5%, at least 10%, at least 20%, at least 30%, at least 40%, at least 50%, or more than 50% of the plasma present in an amount of whole blood used to make the blood fraction.

14. The process of claim 12, wherein the blood fraction contains 5-10%, 10-20%, 20-30%, 20-50%, or 50-100% of the plasma present in an amount of whole blood used to make the blood fraction.

15. The process of claim 12, wherein the blood fraction is the product of a process comprising:
a) optionally, diluting blood with an aqueous solution;
b) centrifuging blood or the diluted blood from step (a) to obtain a cell pellet; and
c) optionally, resuspending the pellet in an aqueous solution, which aqueous solution has the same composition as the aqueous solution of step (a) or has a different composition from the aqueous solution of step (a),
thereby forming the blood fraction.

16. The process of claim 15, comprising a step of forming the blood fraction.

17. The process of claim 8, wherein the sample comprises blood diluted with an aqueous solution.

18. The process of claim 15, wherein the aqueous solution comprises plasma, a cell culture medium, a buffered solution, or a combination thereof.

19. The process of claim 18, wherein the cell culture medium is Roswell Park Memorial Institute (RPMI) medium, Earle medium, or Hanks balanced salt solution.

20. The process of claim 1, wherein the mixture is isotonic to red blood cells.

21. The process of claim 11, wherein the blood is peripheral blood, which is optionally blood from a pregnant female, umbilical cord blood, blood obtained from a subject afflicted with a cancer, or blood obtained from a healthy individual.

22. The process of claim 1, wherein the nucleated cells comprise rare nucleated cells.

23. The process of claim 22, wherein the rare nucleated cells comprise stem cells or cancer cells.

24. The process of claim 22, wherein blood is peripheral blood from a pregnant female and the rare nucleated cells comprise fetal cells.

25. The process of claim 1, further comprising a step of removing all or part of the nucleated cell enriched fraction from the lumen of the container.

26. The process of claim 25, comprising removing all or part of the nucleated cell enriched fraction from the lumen of the container after the mixture separates for 2 to 15 minutes, 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, 5 to 8 minutes.

27. The process of claim 1, further comprising a step of removing all or part of the non-nucleated red blood cell enriched fraction from the lumen of the container.

28. The process of claim 27, comprising removing all or part of the non-nucleated red blood cell enriched fraction from the lumen of the container after the mixture separates for 2 to 15 minutes, 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, 5 to 8 minutes.

29. The process claim 1, wherein the lumen of the container has a fixed volume.

30. The process of claim 1, wherein the lumen of the container comprises a cylindrical section or a polyhedral section.

31. The process of claim 1, wherein the lumen of the container comprises a funnel shaped section.

32. The process of claim 1, wherein the lumen of the container comprises a cylindrical section or polyhedral section joined (a) at the bottom end to a funnel shaped section, (b) at one the top end to an inverted funnel shaped section, or (c) at the bottom end to a funnel shaped section and at the top end to an inverted funnel shaped section.

33. The process of claim 1, wherein container comprises one or more inlet/outlet ports operably connected to the lumen of the container.

34. The process of claim 33, wherein the container further comprises one or more flow deflectors positioned within the lumen of the container to allow for the deflection of a fluid introduced into the lumen of the container through the one or more of the inlet/outlet ports.

35. The process of claim 33, further comprising introducing a heavy liquid into the lumen of the container through a first inlet/outlet port positioned at the bottom of the lumen until all or part of the nucleated cell enriched fraction is forced out of the lumen through a second inlet/outlet port positioned at the top of the lumen.

36. The process of claim 35, wherein an amount of the heavy liquid is present in the lumen of the container during the separation of the mixture.

37. The process of claim 35, wherein the heavy liquid comprises heptacosafluorotributylamine, Ficoll 1.077 g/mL, or Ficoll 1.085 g/mL.

38. The process of claim 36, wherein the mixture is introduced into the lumen of the container after the amount of the heavy liquid is introduced into the container.

39. The process of claim 38, comprising a step of introducing the amount of the heavy liquid into the container before introducing the mixture into the lumen of the container.

40. The process of claim 1, comprising a step of introducing the mixture into the lumen of the container.

41. A nucleated cell enriched fraction obtained by the process of claim 1.

42. A non-nucleated red blood cell enriched fraction obtained by the process of claim 1.

43. A separation device suitable for obtaining the nucleated cell enriched fraction of claim 41.

44. A separation device suitable for obtaining the non-nucleated red blood cell enriched fraction of claim 42.

45. The separation device of claim 43, comprising a container having a lumen and one or more inlet/outlet ports operably connected to the lumen of the container, optionally wherein:
a) the separation device optionally has the features of a device according to FIG. 1; and/or
b) the lumen comprises a cylindrical section with a diameter of 1 to 20 cm, 3 to 8 cm, 4 to 9 cm, 5 to 20 cm, 5 to 10 cm, 6 to 12 cm, 7 to 14 cm, 8 to 12 cm, 8 to 16 cm, 10 to 15 cm, 10 to 20 cm, or a diameter of 5.6 cm.

46. The separation device of claim 45, in which the lumen comprises a cylindrical section and a funnel shaped section.

47. A process for separating nucleated cells from non-nucleated red blood cells, comprising:
a) introducing a mixture comprising nucleated cells, non-nucleated red blood cells, and an aggregating agent into the lumen of a container of a separation device according to claim 45;
b) maintaining the mixture at local gravity until the mixture separates into a nucleated cell enriched fraction and a non-nucleated red blood cell enriched fraction;
c) optionally, recovering one or both fractions; and
d) optionally repeating step (a), step (b) and optionally step (c) one or more times.

48. The process of claim 47, wherein the mixture introduced into the lumen in a volume that reaches a height of 1-4 cm, 1-3 cm, or 1-2 cm in the lumen.

49. The process of claim 48, wherein the mixture introduced into the lumen in a volume that reaches a height of 1.5-2 cm in the lumen.

50. A kit for use in a process for separating nucleated cells from non-nucleated red blood cells, comprising:
a) an aggregating agent and/or a solution comprising an aggregating agent;
b) an aqueous solution;
c) a separation device; or
d) any combination thereof.
